(19) United States
(12) Patent Application Publication
(10) Pub. No.: US 2025/0099195 A1
(43) Pub. Date: Mar. 27, 2025

(54) A CONSOLE SYSTEM FOR A MULTI-ARM ROBOTIC SURGICAL SYSTEM

(71) Applicant: SSI IP HOLDINGS INC., Fort Lauderdale, FL (US)

(72) Inventors: Vishwajyoti Pascual SRIVASTAVA, Gurugram, Haryana (IN); Anil Kumar PATIDAR, Gurugram, Haryana (IN); Sudhir Prem SRIVASAVA, Gurugram, Haryana (IN)

(21) Appl. No.: 18/730,835

(22) PCT Filed: Jan. 28, 2023
(86) PCT No.: PCT/IN2023/050085

§ 371 Date: Jul. 22, 2024

(30) Foreign Application Priority Data
Jan. 28, 2022 (IN) 202211004807

(51) Int. Cl. A61B 34/00; A61B 34/35; A61B 90/00; B25J 9/16; B25J 13/02; B25J 13/04

(52) U.S. Cl. CPC A61B 34/74; A61B 34/71; A61B 90/37; B25J 9/1689; B25J 13/02; B25J 13/04; A61B 34/35

(57) ABSTRACT

A console system (105) for a multi-arm surgical system to increase the ease-of-use and precision of a doctor performing the surgery is described herein. The console system (105) includes a hand controller assembly (207) which comprises a left-hand controller (207a) and a right-hand controller (207b). The hand controller (207b) comprises a backend assembly (401) and a plurality of link members (305) coupled to one another forms a linkage. The plurality of link members comprises an upper elbow (409) or (613) and a lower elbow (411) or (615). The proximal end of the upper elbow (409) is operationally secured to the backend assembly (401) and the distal end of the lower elbow (411) is operationally secured to a gimbal assembly (307). Further, the backend assembly (401) includes a first motorized joint assembly (403), a second motorized joint assembly (405) and a third motorized joint assembly (407) configured to facilitate movement of the hand controller (207b) in x, y and z-axis of a cartesian coordinates.

### TECHNICAL FIELD

The present disclosure generally relates to a console system for a robotic surgical system, and more particularly, to a console system for a surgical robot is designed to increase the ease-of-use and precision of a doctor performing the surgery.

### BACKGROUND

This section is intended to introduce the reader to various aspects of art that may be related to various aspects of the present disclosure, which are described below. This disclosure is believed to be helpful in providing the reader with background information to facilitate a better understanding of the various aspects of the present disclosure. Accordingly, it should be understood that these statements are to be read in this light, and not just as admissions of prior art.

Robotically assisted surgical systems have been adopted worldwide to replace conventional surgical procedures to reduce amount of extraneous tissue(s) that may be damaged during surgical or diagnostic procedures, thereby reducing patient recovery time, patient discomfort, prolonged hospital tenure, and particularly deleterious side effects. In robotically assisted surgeries, the surgeon typically operates a hand controller/master controller/surgeon input device at a surgeon console/console system to seamlessly capture and transfer complex actions performed by the surgeon giving the perception that the surgeon is directly articulating surgical tools/surgical instruments to perform the surgery. The surgeon operating on the surgeon console/console system may be located at a distance from a surgical site or may be located within an operating theatre where the patient is being operated on.

The robotically assisted surgical systems comprises multiple robotic arms aiding in conducting robotic surgeries. The robotically assisted surgical system utilizes a sterile adapter/a sterile barrier to separate non-sterile section of the robotic arm from a mandatory sterile surgical tool/surgical instrument attached to the robotic arm at an operating end. The sterile adaptor/sterile barrier may include a sterile plastic drape that envelops the robotic arm and the sterile adaptor/sterile barrier that operably engages with the sterile surgical tools/surgical instrument in a sterile field.

Traditionally, the console used in the robotically assisted surgical systems are ergonomically inferior due to which the surgeon may feel fatigue during long surgical procedures. Further, the existing consoles are bulky due to which shifting the console between the operating rooms is very difficult.

In the light of aforementioned challenges, there is a need for designing a surgeon console/console system having mobility and ergonomically superior.

### SUMMARY

In one general aspect, a console system (105) may include a hand controller assembly (207), where the hand controller assembly (207) includes a left-hand controller (207a) and a right-hand controller (207b). The console system (105) may also include a viewing means (201) (203), wherein the viewing means (201) is a three-dimensional (3D) monitor, and the viewing means (203) is a two-dimensional (2D) touch screen monitor. The console system (105) may furthermore include an eye tracking camera system (205), wherein the eye tracking camera system (205) is secured to the viewing means (201). The console system (105) may in addition include a foot pedal assembly (209) configured to move inward and outward directions along an axis A-A′, wherein the inward and outward movement is facilitated by a linear actuator (213). The console system may moreover include a telescopic actuator (215) configured to move the hand controller assembly (207), the viewing means (201) (203), the eye tracking camera system (205) in upward and downward direction (along an axis B-B′).

In one general aspect, the hand controller may include a backend assembly (301), wherein the backend assembly (301) facilitate movement of the hand controller (207b) in x, y, and z coordinate axes; and a plurality of link members (305) coupled to one another to form a linkage, wherein a proximal end of the plurality of link members (305) is operationally secured to the backend assembly (301) and a distal of the plurality of link member (305) is operationally secured to a gimbal assembly (307).

In one general aspect, a first motorized joint assembly (403) may include a plate (501). The first motorized joint assembly (403) may also include a first pulley (503), a first spring (505), a first encoder assembly (602), a first cable (506), wherein the first cable (506) circumscribes the first pulley (503) in a counterclockwise direction. Further, the first motorized joint assembly (403) may also include a first capstan (509), where the first capstan (509) is disposed substantially tangential to a circumference of the first pulley (503). The first motorized joint assembly (403) may furthermore include a first motor (507), wherein the first motor (507) is secured to the posterior side of the plate (501). The first motorized joint assembly (403) may in addition include a counterbalance (527), wherein the counterbalance (527) is secured to the posterior side of the plate (501).

In one general aspect, the second motorized joint assembly (405) may include a second pulley (511), a second spring (513), a second encoder assembly (609), a second cable (514), wherein the second cable (514) circumscribes the second pulley (511) in a counterclockwise direction. The second motorized joint assembly (405) may moreover include a second capstan (517), wherein the second capstan (517) is disposed substantially tangential to a circumference of the second pulley (511). The second motorized joint assembly (405) may also include a coupler (601) having a first end (600a) and a second end (600b), wherein the first end (600a) includes a hole (601a), and the second end (600b) includes another hole (601b). The second motorized joint assembly (405) may furthermore include a second motor (515), wherein the second motor (515) is secured to the hole (601a) of the coupler (601). The second motorized joint assembly (405) may in addition include a drive shaft (605).

In one general aspect, third motorized joint assembly (407) may include a third pulley (519), a third spring (521), a third encoder assembly (611), a third cable (522), wherein the third cable (522) circumscribes the third pulley (519) in a counterclockwise direction. The third motorized joint assembly (407) may moreover include a third capstan (525), wherein the third capstan (525) is disposed substantially tangential to a circumference of the third pulley (519). The third motorized joint assembly (407) may also include a coupler (603) having a first end (604a) and a second end (604b), wherein the first end (604a) includes a hole (603a), and the second end (604b) includes another hole (603b). The third motorized joint assembly (407) may furthermore include a third motor (523), wherein the third motor (523) is secured to the hole (603a) of the coupler (603). The third motorized joint assembly (407) may in addition include a drive shaft (607).

### BRIEF DESCRIPTION OF THE DRAWINGS

To further clarify advantages and features of the present disclosure, a more particular description of the disclosure will be rendered by reference to specific embodiments thereof, which is illustrated in the appended drawings. It is appreciated that these drawings depict only typical embodiments of the disclosure and are therefore not to be considered limiting of its scope. The disclosure will be described and explained with additional specificity and detail with the accompanying drawings in which:

FIG. 1 illustrates an example implementation of a multi-arm teleoperated surgical system which can be used with one or more features in accordance with an embodiment of the disclosure;

FIG. 2(a) illustrates a perspective view of an example console system of the multi-arm teleoperated surgical system in accordance with an embodiment of the disclosure;

FIG. 2(b) illustrates a side view of the console system as shown in FIG. 2(a), without side covers in accordance with an embodiment of the disclosure;

FIG. 2(c) illustrates a rear perspective view of a telescopic assembly of the console system in accordance with an embodiment of the disclosure;

FIG. 2(d) illustrates the telescopic assembly as shown in FIG. 2(c), without a viewing means of the console system in accordance with an embodiment of the disclosure;

FIG. 2(e) illustrates an exploded view of a rear portion of the console system in accordance with an embodiment of the disclosure;

FIG. 3 illustrates a perspective view of an example hand controller assembly of the console system in accordance with an embodiment of the disclosure;

FIG. 4 illustrates a perspective view of a backend assembly of the hand controller assembly in accordance with an embodiment of the disclosure;

FIG. 5(a) illustrates a second perspective view of the backend assembly of the hand controller assembly in accordance with an embodiment of the disclosure;

FIG. 5(b) illustrates a rear perspective view of the backend assembly of the hand controller assembly in accordance with an embodiment of the disclosure;

FIG. 6(a) illustrates an exploded view of the backend assembly of the hand controller assembly in accordance with an embodiment of the disclosure;

FIG. 6(b) illustrates second exploded view of the backend assembly of the hand controller assembly in accordance with an embodiment of the disclosure;

FIG. 7(a) illustrates an upper and a lower elbow assembly of the hand controller assembly in accordance with an embodiment of the disclosure;

FIG. 7(b) illustrates an exploded view of the upper and the lower elbow assembly of the hand controller assembly in accordance with an embodiment of the disclosure; and

FIG. 7(c) illustrates second exploded view of the upper and the lower elbow assembly of the hand controller assembly in accordance with an embodiment of the disclosure.

### DETAILED DESCRIPTION OF DISCLOSURE

For the purpose of promoting an understanding of the principles of the disclosure, reference will now be made to the embodiment illustrated in the drawings and specific language will be used to describe the same. It will nevertheless be understood that no limitation of the scope of the disclosure is thereby intended, such alterations and further modifications in the illustrated system, and such further applications of the principles of the disclosure as illustrated therein being contemplated as would normally occur to one skilled in the art to which the disclosure relates.

Any alterations and further modifications to the described console system, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative embodiment can be used or omitted as applicable from other illustrative embodiments. For the sake of brevity, the numerous iterations of these combinations will not be described separately. For simplicity, in some instances the same reference numbers are used throughout the drawings to refer to the same or like parts.

The embodiments below will describe various components of the console system in terms of their state in three-dimensional space. As used herein, the term “position” refers to the location of an object or a portion of an object in a three-dimensional space (e.g., three degrees of translational freedom along Cartesian X, Y, Z coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom).

It will be understood by those skilled in the art that the foregoing general description and the following detailed description are exemplary and explanatory of the disclosure and are not intended to be restrictive thereof. Throughout the patent specification, a convention employed is that in the appended drawings, like numerals denote like components.

Reference throughout this specification to “an embodiment”, “another embodiment”, “an implementation”, “another implementation” or similar language means that a particular feature, structure, or characteristic described in connection with the embodiment is included in at least one embodiment of the present disclosure. Thus, appearances of the phrase “in an embodiment”, “in another embodiment”, “in one implementation”, “in another implementation”, and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures proceeded by “comprises . . . a” does not, without more constraints, preclude the existence of other devices or other sub-systems or other elements or other structures or additional devices or additional sub-systems or additional elements or additional structures.

Unless otherwise defined, all technical and scientific terms used herein have the same meaning as commonly understood by one of ordinary skill in the art to which this disclosure belongs. The apparatus, system, and examples provided herein are illustrative only and not intended to be limiting.

The terms “a” and “an” herein do not denote a limitation of quantity, but rather denote the presence of at least one of the referenced items. Further, the term sterile barrier and sterile adapter denotes the same meaning and may be used interchangeably throughout the description.

Embodiments of the disclosure will be described below in detail with reference to the accompanying drawings.

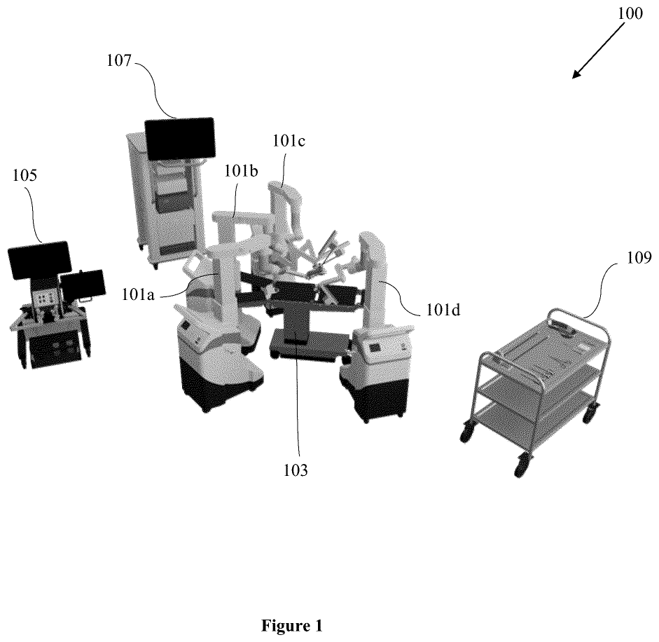

FIG. 1 illustrates an example implementation of a multi-arm teleoperated surgical system which can be used with one or more features in accordance with an embodiment of the disclosure. Specifically, FIG. 1 illustrates the multi-arm teleoperated surgical system (100) having four robotic arms (101a), (101b), (101c), (101d) which are mounted on four robotic arm carts. The four robotic arms (101a), (101b), (101c), (101d) placed on the four robotic arm carts are placed around an operating table (103). The four-robotic arms (101a), (101b), (101c), (101d) as depicted in FIG. 1 is for illustration purposes only and the number of robotic arms may vary depending upon the type of surgery. The robotic arms (101a), (101b), (101c), (101d) may be separately mounted on the four robotic arm carts and the robotic arms (101a), (101b), (101c), (101d) mechanically and/or operationally connected with each other. Alternatively, in other configuration, the robotic arms (101a), (101b), (101c), (101d) may be connected to a central body (which may be single cart) such that the robotic arms (101a), (101b), (101c), (101d) branch out of the central body. Further, the multi-arm teleoperated surgical system (100) may include a console system (105), a vision cart (107) and a surgical instrument and accessory table (109).

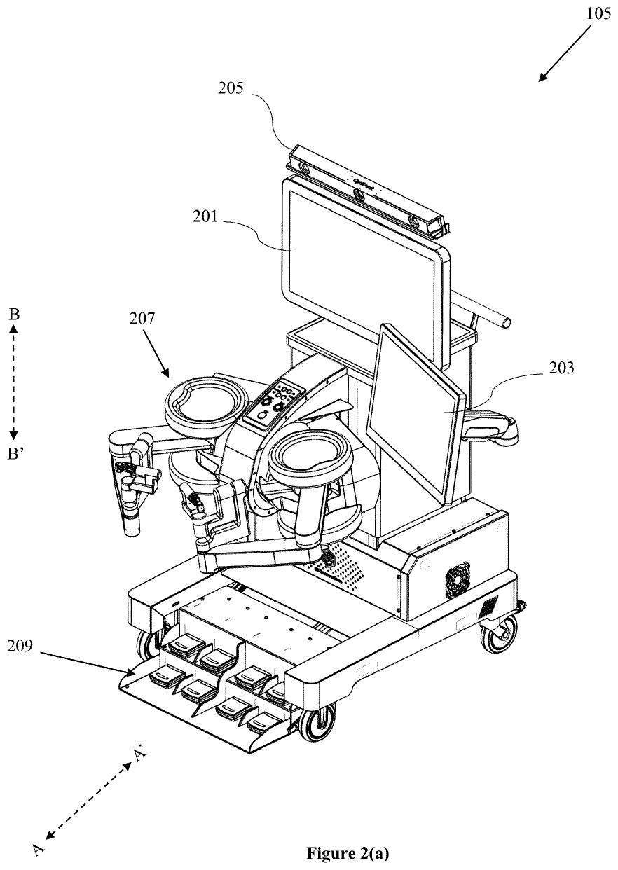

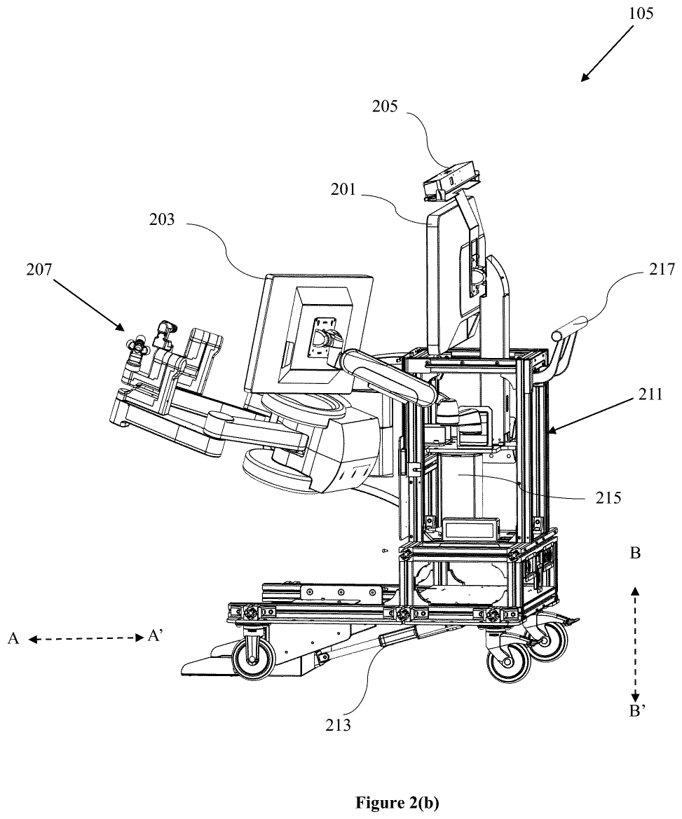

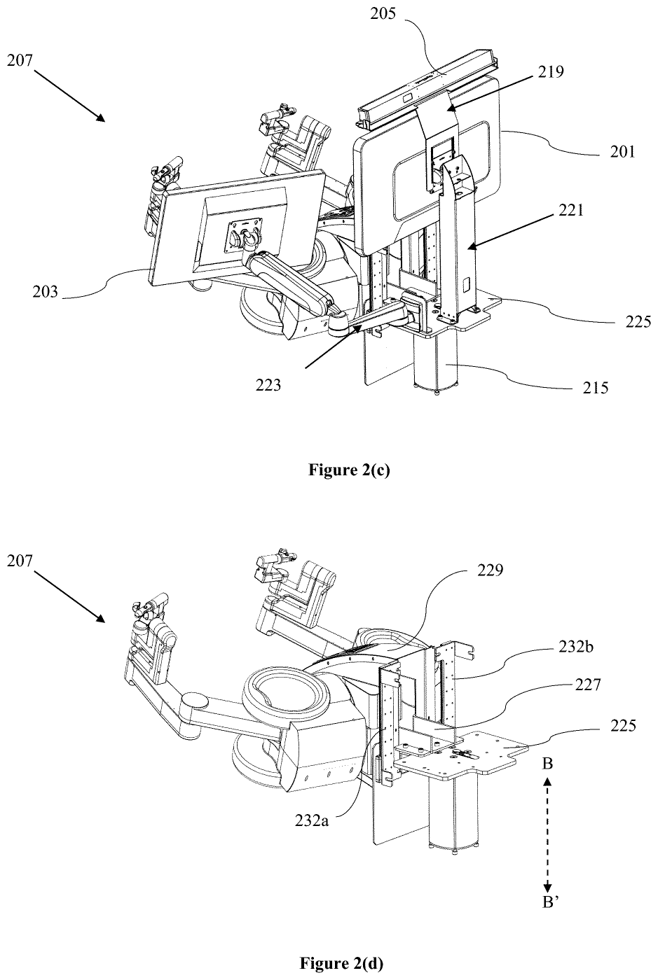

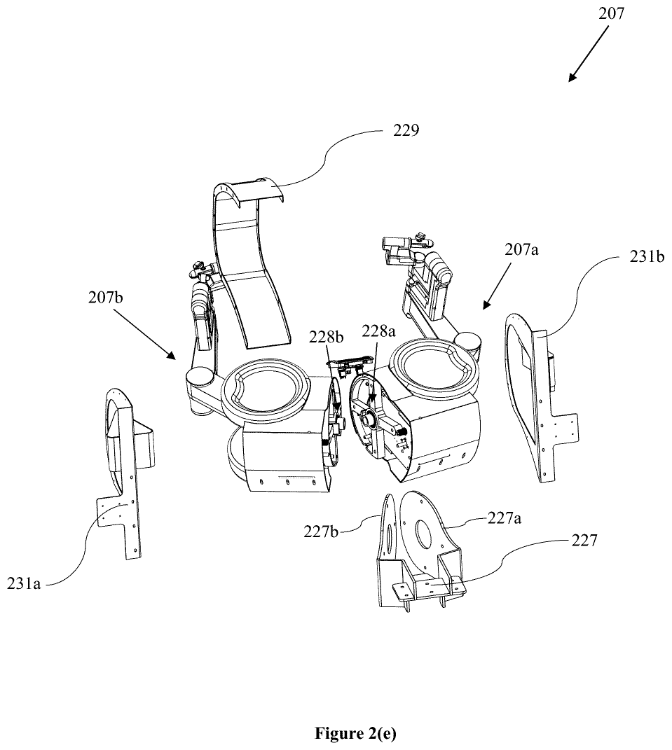

FIG. 2(a) illustrates a perspective view of an example console system of the multi-arm teleoperated surgical system in accordance with an embodiment of the disclosure. The console system (105) aids a surgeon to remotely operate a patient lying on the operating table (103) by controlling various surgical instruments and an endoscope which are mounted on any of the robotic arms (101a), (101b), (101c), (101d) (as shown in FIG. 1). The surgeon console/console system (105) may be configured to control the movement of the surgical instruments mounted in any of the robotic arms (101a), (101b), (101c), (101d). The console system (105) may comprise of an adjustable viewing means (201) and (203), but not limited to 2D/3D monitors, wearable viewing means (not shown), immersive viewing means (not shown) and in combination thereof. The console system (105) may be equipped with multiple displays which would not only show the 3D high definition (HD) endoscopic view of a surgical site but may also shows additional information from various medical equipment's which surgeon may need during the surgery. Further, the viewing means (201) and (203) may provide various modes of the multi-arm teleoperated surgical system (100), but not limited to identification of number of robotic arms attached, current surgical instruments type attached, current instruments end effector tip position, collision information, medical data like ECG, ultrasound display, fluoroscopic images, CT, MRI information and the like. The viewing means (201) may be a 3D monitor and the viewing means (203) may be a 2D touch screen monitor.

The console system (105) may further comprise of an eye tracking camera system (205) for detecting direction of the surgeon's eye gaze and accordingly activates/deactivates the surgical instruments controls from the surgeon. Furthermore, the console system (105) may comprise of a hand controller assembly (207), one or more foot pedal assembly (209). A linear actuator (213) is mounted on an inferior end of the console system (105) which is configured to facilitate the movement of a foot pedals assembly (209) “inwardly” and “outwardly” along the axis A-A′. The foot pedals assembly may also be configured to move upward and downward directions along an axis B-B′. The hand controller assembly (207) at the console system (105) may require to seamlessly capture and transfer complex actions performed by the surgeon giving the perception that the surgeon is directly articulating the surgical instruments inside the patient body. In some embodiments, the hand controller assembly (207) may be one or more manually operated input devices, such as a joystick, exoskeletal glove, a powered and gravity-compensated manipulator, or the like. The surgeon may sit on a resting apparatus such as a chair (not shown) in proximity to the console system (105) such that the surgeon's arms may rest on an arm rest, while controlling the hand controller assembly (207). The chair may be adjustable with means in height, elbow rest and the like according to the ease of the surgeon. Also, various control means may be provided on the chair and the arm rest. Further, the console system (105) may be at one location inside an operation theatre or may be placed at any other location in the hospital provided connectivity to the robotics arms (101a, 101b, 101c, 101d) via wired or wireless means is maintained.

In some implementations, the viewing means (201) and (203), the eye tracking camera system (205) and the hand controller assembly (207) may be configured to move in “upward” and “downward” direction (along an axis B-B′) based on the surgeon height and body habitus. Further, the viewing means (201) and (203), the eye tracking camera system (205) and the hand controller assembly (207) may be configured to move independently in “upward” and “downward” direction (along the axis B-B′) based on the surgeon height and body habitus. For example, a telescopic actuator (shown in FIGS. 2(b), (c) and (d)) may be configured in a manner to facilitate the movement of the viewing means (201) and (203), the eye tracking camera system (205) and the hand controller assembly (207) together in the “upward” and “downward” direction based on the surgeon height and the body habitus. In another example, plurality of telescopic actuators (not shown) can be used to independently facilitate the movement of the viewing means (201) and (203), the eye tracking camera system (205) and the hand controller assembly (207) in the “upward” and “downward” direction based on the surgeon height and the body habitus.

Now referring together, FIGS. 2(a), 2(b), 2(c), 2(d) and 2(e), the console system (105) includes a handle (217) for transporting the console system (105). The handle (217) may be secured on a superior end of a frame structure (211). The console system (105) may include a lifting means such as a telescopic actuator (215) which may facilitate the movement of the viewing means (201) and (203), the eye tracking camera system (205) and the hand controller assembly (207) together in the “upward” and “downward” direction based on the surgeon height and the body habitus.

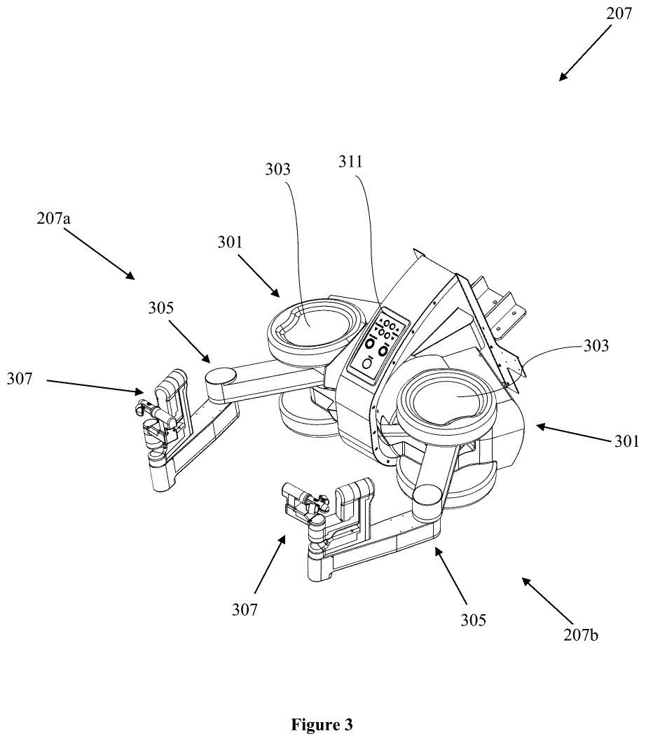

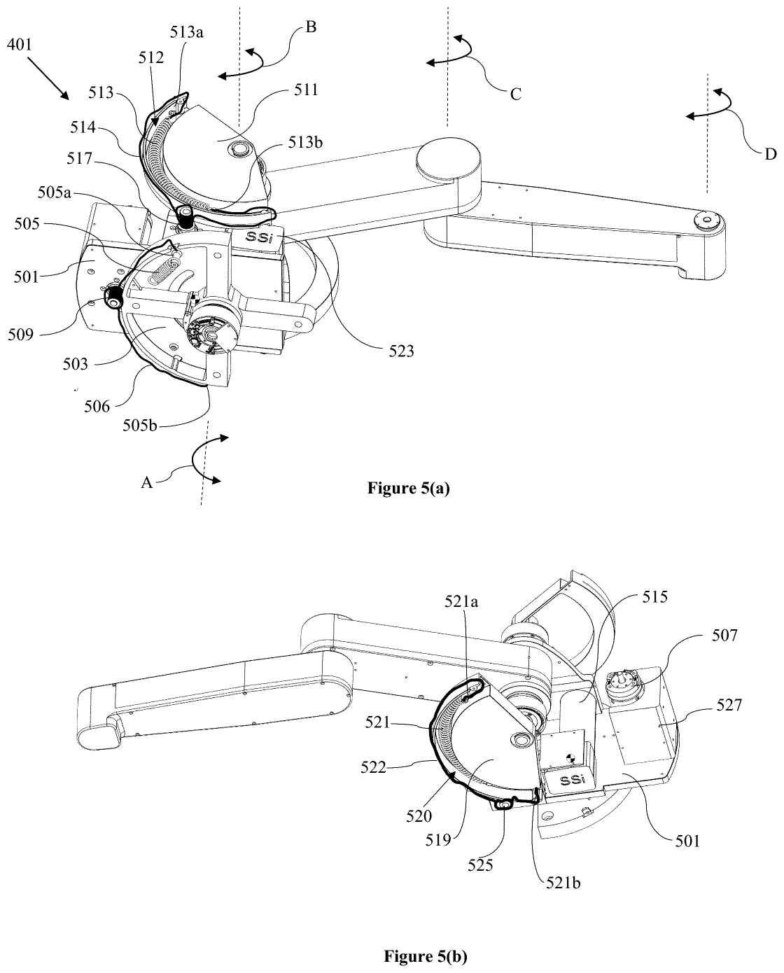

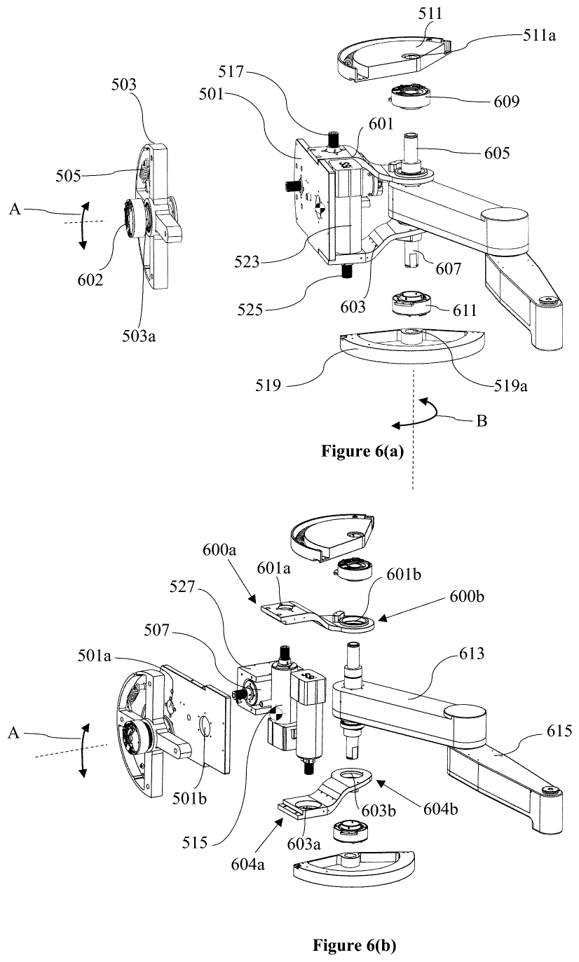

In some implementations, an eye tracking camera mount (219) is secured to the viewing means (201) at one end and another end is secured to the eye tracking camera system (205). A linear mount (221) may be secured to the viewing means (201) at one end and another end is secured to a base mount structure (225). A link (223) is secured to the viewing means (203) at one end and another end is secured to the base mount structure (225). Further, a mount bracket (227) at one end is secured to the base mount structure (225) and another end is secured to the hand controller assembly (207). The hand controller assembly (207) may be a left-hand controller (207a) and a right-hand controller (207b) (considering viewing from front side as shown in FIG. 3). The left-hand controller (207a) at its proximal end includes a pulley (228a) and a right-hand controller (207b) at its proximal end includes a pulley (228b) (the pulley is described in detail in explanation of FIG. 5-6). The mount bracket (227) includes a plurality of flanges such as first flange (227a) and a second flange (227b). The flange (227a) is secured to the left-hand controller (207a) through a pulley (228a). More specifically, the flange (227a) is screwed to the pulley (228a). The flange (227b) is secured to the right-hand controller (207b) through a pulley (228b). More specifically, the flange (227b) is screwed to the pulley (228b). As the left-hand controller (207a) and the right-hand controller (207b) is secured to the mount bracket (227) (through the flanges (227a), (227b) and the pulleys (228a), (228b)), and further, the mount bracket (227) is secured to the base mount structure (227), therefore, the telescopic actuator (215) lifting the base mount structure (227) in “upward” and “downward” directions along the axis B-B′ will also lift the left hand controller (207a) and the right hand controller (207b).

A side cover (231a), (231b) is secured to a rail mount (232a), (232b) respectively. A top cover (229) is secured to the side cover (231a), (231b). The base mount structure (225) is secured to an upper end of the telescopic actuator (215) at one end to facilitate the movement of the viewing means (201) and (203), the eye tracking camera system (205) and the hand controller assembly (207) in “upward” and “downward” directions along the axis B-B′.

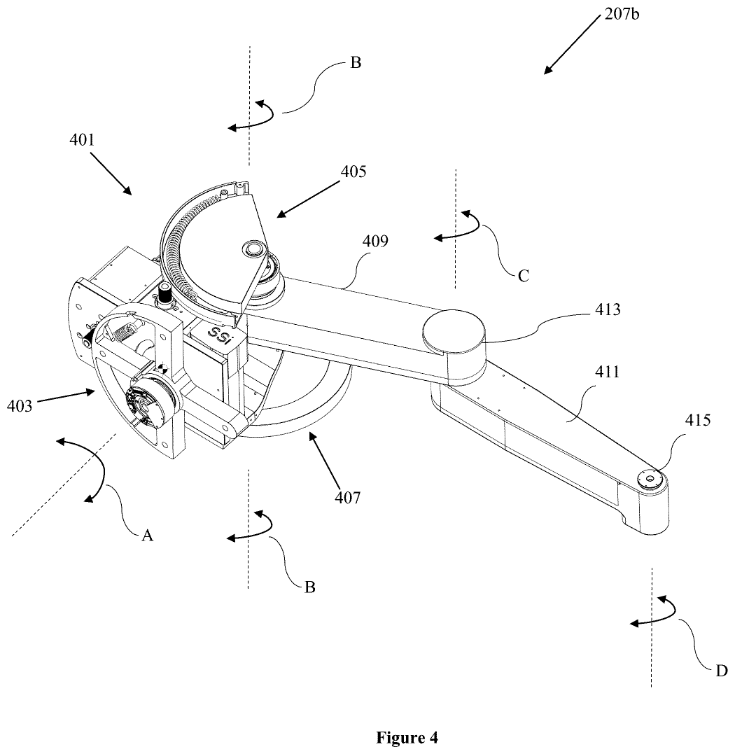

FIG. 3 illustrates a perspective view of an example hand controller assembly of the console system in accordance with an embodiment of the disclosure. The hand controller assembly (207) includes a left-hand controller (207a) and a right-hand controller (207b). The left-hand controller (207a) and the right-hand controller (207b) are similar in functionality, dimensions, and components. Henceforth, in the description, only one hand controller is described, and it should not be considered as a limitation that the same explanation may not be applied to the other hand controller. More specifically, FIG. 4 to FIG. 7 illustrates right-hand controller (207b) and the same explanation will also be applicable for left-hand controller (207a) without any limitations.

In an embodiment, the hand controller assembly (207) may comprise a backend assembly (301) to which a backend cover (303) is mounted to cover the backend assembly (301), plurality of link members (305) are coupled to one another forming a linkage. A proximal end of the plurality of link member (305) is operationally secured to the backend assembly (301) and a distal end of the plurality of link member (305) is operationally secured to a gimbal assembly (307). A switch panel (311) is mounted on the top cover (229) (as shown in FIG. 2(e)). The switch panel (311) comprises a multiple switch but not limited to power ON/OFF, emergency STOP, motion control and height adjustments of viewing means (201) and (203), the eye tracking camera system (205) and the hand controller assembly (207).

FIG. 4 illustrates a perspective view of a backend assembly of a hand controller in accordance with an embodiment of the disclosure. The proximal end of the hand controller (207b) includes a backend assembly (401). The backend assembly (401) includes a plurality of motorized joints to facilitate a translational movement of the hand controller (207b) in any of the x, y and z axis of a cartesian coordinates. The plurality of motorized joints may include a capstan and pulley mechanism to achieve the desired translational movement of the hand controller (207b) in x, y and z axis of the cartesian coordinates. In a specific embodiment, a first motorized joint assembly (403) may facilitate the movement of the hand controller (207b) in z-axis of the cartesian coordinates (upward & downward) by rotating the first motorized joint assembly (403) along an axis of rotation ‘A’. A second motorized joint assembly (405) may facilitate the movement of the hand controller (207b) in x-axis of the cartesian coordinates (right & left) by rotating the second motorized joint assembly (405) along an ais of rotation ‘B’. A third motorized joint assembly (407) may facilitate the movement of the hand controller (207b) in y-axis of the cartesian coordinates (in and out) by rotating the third motorized joint assembly (407) along an ais of rotation ‘B’. The hand controller (207b) may include a plurality of link members (305) (as shown in FIG. 3) such as an upper elbow (409) and a lower elbow (411). A proximal end of the upper elbow (409) is operationally secured to the backend assembly (401) and a distal end of the upper elbow (409) is operationally secured to a proximal end of the lower elbow (411) at a rotational joint (413). Further, the proximal end of the lower elbow (411) which is secured to the distal end of the upper elbow (409) is configured to rotate at an axis of rotation ‘C’. A distal end of the lower elbow (411) is secured to the gimbal assembly (307) (as illustrated in FIG. 3) at a rotational joint (415). The upper elbow (409) at its proximal end is capable of rotating at the axis of rotation ‘B’.

In an example embodiment, the gimbal assembly (307) is secured to a rotational joint (415). The gimbal assembly (307) is configured for rotating at the axis of rotation ‘D’. The surgeon, while performing surgery, maneuvers the gimbal assembly (307) (as shown in FIG. 3) in various directions and orientations. The translational motion of the surgeon hand in x, y and z-axes of the cartesian coordinates (which will translate in the movement of the robotic arms 101a-101d as shown in FIG. 1) while holding the gimbal assembly (307) is achieved by the rotational movement of the first motorized joint assembly (403), the second motorized joint assembly (405) and the third motorized joint assembly (407). The rotational movement of the surgeon hand which will translate to the distal end of the surgical instruments such as pitch, yaw, roll is achieved by the gimbal assembly (307).

In an embodiment, when the surgeon holds the gimbal assembly (307) (as shown in FIG. 3) and tries to move his/her hand substantially in ‘upward’ and/or ‘downward’ directions (+z and −z axis of the cartesian coordinates), a motion translates from the lower elbow (411) and the upper elbow (409) to facilitate the rotation of the first motorized joint assembly (403) in the axis of rotation ‘A’. At the same time, the second motorized joint assembly (405) and the third motorized joint assembly (407) remains substantially stationary and also, the rotational joint (413) and (415) remains substantially stationary when the surgeon tries to move his/her hand in substantially in ‘upward’ and/or ‘downward’ directions (+z and −z-axis of the cartesian coordinates).

In a specific embodiment, when the surgeon holds the gimbal assembly (307) and tries to move his/her hand purely in “upward” and/or “downward” directions (+z and −z axis of the cartesian coordinates), a motion translates from the lower elbow (411) and the upper elbow (409) to facilitate the rotation of the first motorized joint assembly (403) in the axis of rotation “A”. At the same time, the second motorized joint assembly (405) and the third motorized joint assembly (407) remains completely stationary and also, the rotational joint (413) and (415) remains completely stationary when the surgeon tries to move his/her hand in substantially in “upward” and/or “downward” directions (+z and −z axis of the cartesian coordinates).

Further, when the surgeon holds the gimbal assembly (307) and tries to move his/her hand substantially in “right” and/or “left” directions (+x and −x axis of the cartesian coordinates), a motion translates from the lower elbow (411) and the upper elbow (409) to facilitate the rotation of the second motorized joint assembly (405) in the axis of rotation ‘B’. The first motorized joint assembly (403) and the third motorized joint assembly (407) remains substantially stationary. At the same time, the first motorized joint assembly (403) and the third motorized joint assembly (407) remains substantially stationary and also, the rotational joint (413) and (415) remains substantially stationary when the surgeon tries to move his/her hand in substantially in “right” and/or “left” directions (+x and −x axis of the cartesian coordinates).

In a specific embodiment, when the surgeon holds the gimbal assembly (307) and tries to move his/her hand purely in “right” and/or “left” directions (+x and −x axis of the cartesian coordinates), a motion translates from the lower elbow (411) and the upper elbow (409) to facilitate the rotation of the second motorized joint assembly (405) in the axis of rotation “B”. The first motorized joint assembly (403) and the third motorized joint assembly (407) remains completely stationary. At the same time, the first motorized joint assembly (403) and the third motorized joint assembly (407) remains completely stationary and also, the rotational joint (413) and (415) remains completely stationary when the surgeon tries to move his/her hand purely in “right” and/or “left” directions (+x and −x axis of the cartesian coordinates).

Furthermore, when the surgeon holds the gimbal assembly (307) and tries to move his/her hand substantially in “in” and “out” directions (+y and −y axis of the cartesian coordinates), only the lower elbow (411) participates in the movement to facilitate the rotation of the third motorized joint assembly (407) in the axis of rotation “B”. The first motorized joint assembly (403) and the second motorized joint assembly (405) remains substantially stationary. At the same time, the rotational joint (415) remains fixed, and the rotational joint (413) rotates along the axis of rotation “C” when the surgeon tries to move his/her hand in substantially “in” and “out” directions.

In a specific embodiment, when the surgeon holds the gimbal assembly (307) and tries to move his/her hand purely in “in” and “out” directions (+y and −y axis of the cartesian coordinates), only the lower elbow (411) participates in the movement to facilitate the rotation of the third motorized joint assembly (407) in the axis of rotation “B”. The first motorized joint assembly (403) and the second motorized joint assembly (405) remains completely stationary. At the same time, the rotational joint (415) remains fixed, and the rotational joint (413) rotates along the axis of rotation “C” when the surgeon tries to move his/her hand in purely “in” and “out” directions.

The surgeon, while performing the surgery may maneuver the hand controller (207b) (as shown in FIG. 3) in different directions and orientations while holding the gimbal assembly (307). The hand controller (207b) (as shown in FIG. 3) may be maneuvered in the x, y and z axes of the cartesian coordinates such that the motion of the hand controller (207b) (as shown in FIG. 3) in x, y and z directions may be dynamic within a workspace of the hand controller (207b) (as shown in FIG. 3). To achieve the dynamic movement of the hand controller in the x, y, and z directions, all the three motorized joint assemblies (403, 405 and 405) may participate/rotate simultaneously.

As illustrated in FIGS. 5(a) and 5(b), the first motorized joint assembly (403) (as explained in FIG. 4), the backend assembly (401) comprises a first pulley (503) which may be secured to an exterior side of a plate (501). The first pulley (503) may be in D shape with a protruding profile from center of a diameter such that the protruding profile is at right angle with the diameter (as shown in FIG. 5(a)). Alternatively, the first pulley (503) may be in circular or partially circular shape or in any other shape, as may be required. The first pulley (503) may be either of a segmented construction, as shown in FIG. 5(a), or solid, or perforated, or any other construction, as may be required. The first pulley (503) may include a first spring (505) in a recess of the segmented construction at the exterior side. A first cable (506) is secured to one end (505a) of the first spring (505) and the said first cable (506) circumscribes the first pulley (503) in a counterclockwise direction. In other implementations, the first cable (506) may circumscribe the first pulley (503) in a clockwise direction based on the design requirements. The first cable (506) wraps a first capstan (509) (the first capstan (509) disposed substantially tangential to the circumference of the first pulley (503)) several times and secured to another end (505b) of the first pulley (503). The first capstan (509) is mounted to a first motor (507) to facilitate the rotational movement of the first motorized joint assembly (403) (as explained in FIG. 4) along the axis of rotation ‘A’.

Further, the backend assembly (401) comprises a second pulley (511). The second pulley (511) may be in substantially D shaped (as shown in FIG. 5(a)) or alternatively, or circular or partially circular shape. The second pulley (511) at its exterior surface around the circumference includes a recess (512) and a second spring (513) is disposed within the recess (512). A second cable (514) is secured to one end (513a) of the second spring (513) and the said second cable (514) circumscribes the second pulley (511) in counterclockwise direction. In other implementations, the said second cable (514) may circumscribe the second pulley (511) in clockwise direction based on the design requirement. The second cable (514) wraps to a second capstan (517) (the second capstan (517) disposed substantially tangential to the circumference of the second pulley (511)) several times and secured to another end (513b) of the second pulley (511). The second capstan (517) is mounted to a second motor (515) to facilitate the rotational movement of the second motorized joint assembly (405) (as explained in FIG. 4) along the axis of rotation ‘B’.

Furthermore, the backend assembly (401) comprises a third pulley (519). The third pulley (519) may be substantially D shaped (as shown in FIG. 5(a)) or alternatively, circular or partially circular shape. The third pulley (519) at its interior surface around the circumference includes a recess (520) and a third spring (521) is disposed within the recess (520). A third cable (522) is secured to one end (521a) of the third spring (521) and the said third cable (522) circumscribes the third pulley (519) in counterclockwise direction. In other implementations, the said third cable (522) circumscribes the third pulley (519) in clockwise direction based on the design requirements. The third cable (522) is wrapped to a third capstan (525) (the third capstan (525) disposed substantially tangential to the circumference of the third pulley (519)) several times and secured to another end (521b) of the third pulley (519). The third capstan (525) is mounted to a third motor (523) to facilitate the rotation movement of the third motorized joint assembly (407) (as explained in FIG. 4) along the axis of rotation “B”.

Now referring to FIGS. 6(a) and (b), some components in FIGS. 6(a) and (b) are not labelled and the same can be referred to from the referral numerals of the previous figures. The upper elbow may interchangeably be refereed by the referral numerals (409) or (613) and the lower elbow may interchangeably be refereed by the referral numerals (411) and (615). The first motorized joint assembly (403) (as explained in FIGS. 4, 5(a) and 5(b)) includes the plate (501) (the plate (501) is common in all the three motorized joint assemblies (403), (405) and (407)), the first pulley (503), wherein a circular profile (503a) disposed substantially to a center of a diameter of the first pulley (503), the first spring (505), the first cable (506), the first capstan (509), the first motor (507) and a first encoder assembly (602). The plate (501) includes plurality of holes to accommodate the first capstan (509), the first encoder assembly (602) and for screwing/fastening the first pulley (503) to the plate (501). In a specific embodiment, the plate (501) at its horizontal end, includes a hole (501a) and a hole (501b). The hole (501a) at a first horizontal end of the plate (501) may accommodate the first capstan (509) and the hole (501b) at a second horizontal end of the plate (501) may accommodate the first encoder assembly (602). The first motor (507) is secured to a posterior side of the plate (501) toward the hole (501a) and the first capstan (509) configured to the first motor (507) protrudes through the hole (501a). A counterweight (527) covering the first motor (507) is also secured to the posterior side of the plate (501), around the hole (501a). The hole (501b) may also be configured to secure the first encoder assembly (602), the first pulley (503) to the plate (501) in the same axis so as to facilitate the rotational movement of the first motorized joint assembly (403) along the axis of rotation ‘A’.

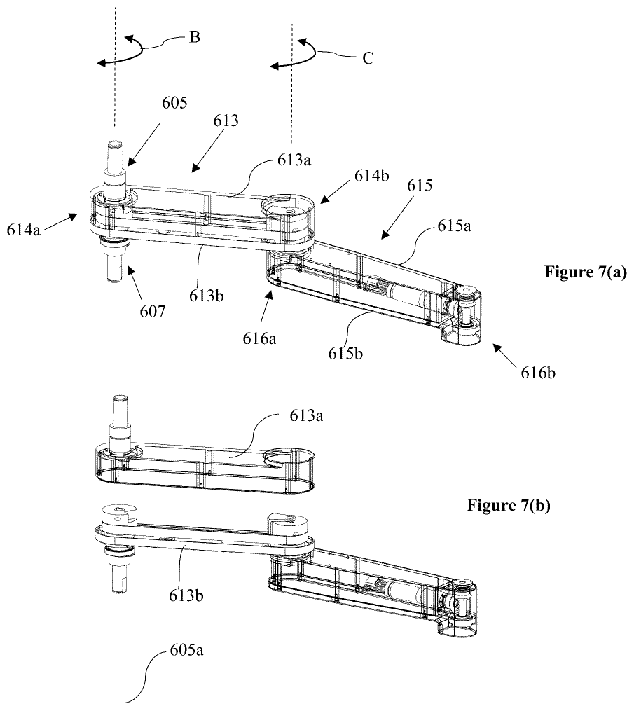

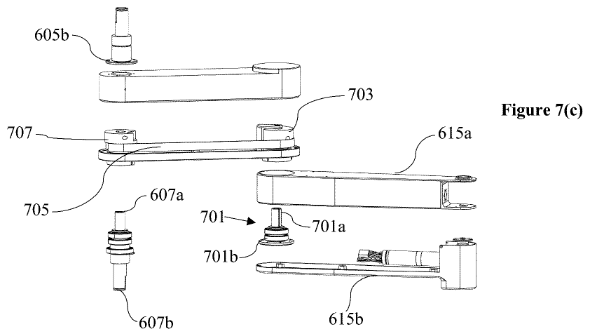

The second motorized joint assembly (405) (as explained in FIGS. 4, 5(a) and 5(b)) includes the plate (501) (the plate (501) is common in all the three motorized joint assemblies (403), (405) and (407)), the second pulley (511) and a circular profile (511a) disposed substantially to a center of a diameter of the second pulley (511). Further, the second motorized joint assembly (405) includes a coupler (601) having a first end (600a) and a second end (600b) wherein, the first end (600a) includes a hole (601a), and the second end (600b) includes another hole (601b). Furthermore, the second motorized joint assembly (405) includes the second motor (515) secured to the hole (601a) of a coupler (601). The first end (600a) of the coupler (601) is further secured to the posterior side of the plate (501) and the second end (600b) of the coupler (601) is secured to the proximal end of the upper elbow (613) (at the upper elbow upper link (613a) as shown in FIG. 7(a)) by bearing and a drive shaft (605). The circular profile/the hole (601a) at the first end (600a) of the coupler (601) is configured to accommodate the second capstan (517), wherein the second capstan (517) is secured to one end of the second motor (515). The circular profile/the hole (601b) at the second end (600b) of the coupler (601) may be bigger than the circular profile/the hole (601a) at the first end (600a) of the coupler (601). A drive shaft (605) at its lower end is secured to the superior side of the upper elbow (613) (As shown in FIG. 7(a), the drive shaft (605) lower end is secured to the upper elbow upper link (613a)). The rotation of the drive shaft (605) is identified by a second encoder assembly (609). The second pulley (511), the circular profile (511a) are placed in the vertical axis of the drive shaft (605). More specifically, the circular profile (511a), the second encoder assembly (609) and the hole (601b) of the coupler (601) are placed/secured in the vertical axis of the drive shaft (605).

The third motorized joint assembly (407) (as explained in FIGS. 4, 5(a) and 5(b)) includes the plate (501) (the plate (501) is common in all the three motorized joint assemblies (403), (405) and (407)), the third pulley (519), a circular profile (519a) disposed substantially to the center of a diameter of the third pulley (519). Further, the third motorized joint assembly (407) includes a coupler (603) having a first end (604a) and a second end (604b), wherein the first end (604a) includes a hole (603a), and the second end (604b) includes another hole (603b). Furthermore, the third motorized joint assembly (407) includes the third motor (523) secured to the hole (603a) of a coupler (603). The first end (604a) of the coupler (603a) is further secured to the posterior side of the plate (501) and the second end (604b) of the coupler (603) is secured to proximal end of the upper elbow (613) (at the upper elbow lower link (613b) as shown in FIG. 7(a)) by bearing and drive shaft (607). The circular profile/the hole (603a) at the first end (604a) of the coupler (603) is configured to accommodate the third capstan (525), wherein the third capstan (525) is secured to one end of the third motor (523). The circular profile/the hole (603b) at the second end (604b) of the coupler (603) may be bigger than the circular profile/the hole (603a) at the first end (604a) of the coupler (603). A drive shaft (607) at its upper end is secured to the inferior side of the proximal end of the upper elbow (613) (As shown in FIG. 7(a), the drive shaft (607) upper end is secured to the upper elbow lower link (613b)). The rotation of the drive shaft (607) is identified by a third encoder assembly (611). The third pulley (519), the circular profile (519a) are placed in the vertical axis of the drive shaft (607). More specifically, the circular profile (519a), the third encoder assembly (611) and the second end (604b) of the coupler (603) are placed in the vertical axis of the drive shaft (607).

Referring to FIGS. 7(a), (b) and (c), the upper elbow (613) comprises an upper elbow proximal end (614a) and an upper elbow distal end (614b). Similarly, the lower elbow (615) comprises a lower elbow proximal end (616a) and a lower elbow distal end (616b). Further, the upper elbow (613) comprises an upper elbow upper link (613a) and an upper elbow lower link (613b) which are secured together. Similarly, the lower elbow (615) comprises a lower elbow upper link (615a) and a lower elbow lower link (615b) which are secured together.

The drive shaft (605) comprises a drive shaft upper end (605a) and a drive shaft lower end (605b), wherein the drive shaft lower end (605b) comprises of a circular profile. The circular profile of the drive shaft lower end (605b) is secured towards the upper elbow proximal end (614a) at the upper elbow upper link (613a). The rotation of the upper elbow (613) along the axis of rotation ‘B’ also facilitate rotation of the drive shaft (605), without rotating the drive shaft (607).

The drive shaft (607) comprises a drive shaft upper end (607a) and a drive shaft lower end (607b). The drive shaft upper end (607a) is secured towards the upper elbow proximal end (614a) at the upper elbow lower link (613b). The rotation of the upper elbow (613) along the axis of rotation ‘B’ does not facilitate rotation of the drive shaft (607) or the rotation of the upper elbow (613) is independent to the rotation of the drive shaft (607).

According to an embodiment, when the surgeon holds the gimbal assembly (307) and tries to move his/her hand substantially and/or purely in “right” and/or “left” directions (+x and −x axis of the cartesian coordinates), a motion translates from the lower elbow (615) and the upper elbow (613) to facilitate the rotation of the drive shaft (605) in the axis of rotation ‘B’. As the drive shaft (605) is rotationally secured the second encoder assembly (609) and the second pulley (511). Therefore, the rotation of the drive shaft (605) facilitate the rotation of the second motorized joint assembly (405) along the axis of rotation ‘B’.

Now referring to FIG. 7(c), the drive shaft (701) comprises a drive shaft upper end (701a) and a drive shaft lower end (701b), wherein the drive shaft lower end (701b) comprises of a circular profile. The circular profile of the drive shaft lower end (701b) is secured towards the lower link proximal end (616a) at the lower elbow lower link (615b). The rotation of the lower elbow (615) along the axis of rotation ‘C’ also facilitate rotation of the drive shaft (701). A pulley (707) is disposed towards the proximal end (614a) of the upper elbow (613) at the upper elbow lower link (613b) and a pulley (703) is disposed towards the upper elbow distal end (614b) of the upper elbow (613) upper elbow lower link (613b). A drive belt (705) is disposed from the pulley (707) to the pulley (703). Further, the drive shaft upper end (701a) is secured to the pulley (703) and the pulley (703) is rotationally connected to the drive belt (705) such that the rotation of the drive shaft (701) along the axis of rotation ‘C’ will also facilitate the rotation of the pulley (703). Further, the rotation of the pulley (703) will further facilitate the rotation of the pulley (707) as the pulley (703) and the pulley (707) are rotationally connected to the drive belt (705). The drive shaft upper end (607a) is secured to the pulley (707) towards the upper elbow proximal end (614a) at the upper elbow lower link (613b). The rotation of the pulley (707) at the axis of rotation ‘B’ will facilitate the rotation of the drive shaft (607).

A drive belt (705) at its one end is mounted to the pulley (703) and other side is mounted a pulley (707). The pulley (707) is operationally mounted to the upper end of the drive shaft (607) at its vertical axis to provide a circulation motion of the drive shaft (607). As the lower end of the drive shaft (607) is secured to the pulley (707), therefore, the drive shaft (607) facilitates the rotation of the third pulley (519) which in turn actuates the third motor (523) to facilitate the smooth motion by rotating the third capstan (525) with the help of the second cable (514).

According to an embodiment, when the surgeon holds the gimbal assembly (307) and tries to move his/her hand substantially and/or purely in “in” and/or “out” directions (+y and −y axis of the cartesian coordinates), a motion translates from the lower elbow (615) to facilitate the rotation of the drive shaft (607) in the axis of rotation ‘B’. As the drive shaft (607) is rotationally secured the third encoder assembly (611) and the third pulley (519). Therefore, the rotation of the drive shaft (607) facilitate the rotation of the third motorized joint assembly (407) along the axis of rotation ‘B’.

The foregoing descriptions of exemplary embodiments of the present disclosure have been presented for purposes of illustration and description. They are not intended to be exhaustive or to limit the disclosure to the precise forms disclosed, and obviously many modifications and variations are possible in light of the above teaching. The exemplary embodiment was chosen and described in order to best explain the principles of the disclosure and its practical application, to thereby enable others skilled in the art to best utilize the disclosure and various embodiments with various modifications as are suited to the particular use contemplated. It is understood that various omissions, substitutions of equivalents are contemplated as circumstance may suggest or render expedient but is intended to cover the application or implementation without departing from the spirit or scope of the claims of the present disclosure.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

While specific language has been used to describe the disclosure, any limitations arising on account of the same are not intended. As would be apparent to a person in the art, various working modifications may be made to the apparatus in order to implement the inventive concept as taught herein.

1. A console system (105) for a multi-arm robotic surgical system, the console system (105) comprising:
a hand controller assembly (207), wherein the hand controller assembly (207) includes a left-hand controller (207a) and a right-hand controller (207b);
a viewing means (201) (203), wherein the viewing means (201) is a three-dimensional (3D) monitor, and the viewing means (203) is a two-dimensional (2D) touch screen monitor;
an eye tracking camera system (205), wherein the eye tracking camera system (205) is secured to the viewing means (201);
a foot pedal assembly (209) configured to move inward and outward directions along an axis A-A′, wherein the inward and outward movement is facilitated by a linear actuator (213); and
a telescopic actuator (215) configured to move the hand controller assembly (207), the viewing means (201) (203), the eye tracking camera system (205) in upward and downward direction (along an axis B-B′).

2. The console system (105) as claimed in claim 1, wherein the left-hand controller (207a) at its proximal end includes a pulley (228a) and the right-hand controller (207b) at its proximal end includes a pulley (228b).

3. The console system (105) as claimed in claim 1, further comprising:
a frame structure (211);
a base mount structure (225) secured to an upper end of the telescopic actuator (215);
a mount bracket (227) secured to the base mount structure (225) at one end and another end is secured to the hand controller assembly (207);
a linear mount (221) secured to the viewing means (201) at one end and another end is secured to the base mount structure (225); and
a link (223), secured to the viewing means (203) at one end and another end is secured to the base mount structure (225).

4. The console system (105) as claimed in claim 3, wherein the linear actuator (215) facilitate the movement of the base mount structure (225) in upward and downward direction (along an axis B-B′).

5. The console system (105) as claimed in claim 3, the mount bracket (227) comprises a plurality of flanges (227a), (227b), wherein the flange (227a) is secured to a left-hand controller (207a) and the flange (227b) is secured to the right-hand controller (207b).

6. A hand controller (207b) of a console system (105), comprising:
a backend assembly (301), wherein the backend assembly (301) facilitate movement of the hand controller (207b) in x, y, and z coordinate axes; and
a plurality of link members (305) coupled to one another to form a linkage, wherein a proximal end of the plurality of link members (305) is operationally secured to the backend assembly (301) and a distal of the plurality of link member (305) is operationally secured to a gimbal assembly (307).

7. The hand controller (207b) of the console system (105) as claimed in claim 6, wherein the plurality of link members (305) further comprising:
an upper elbow (409) (or 613), having an upper elbow upper link (613a) and an upper elbow lower link (613b), secured together; and
a lower elbow (411) (or 615), having a lower elbow upper link (615a) and a lower elbow lower link (615b), secured together, wherein a upper elbow distal end (614b) is rotationally secured to a lower elbow proximal end (616a).

8. The hand controller (207b) as claimed in claim 7, wherein a drive shaft (605) comprises a drive shaft upper end (605a) a drive shaft lower end (605b), and the drive shaft lower end (605b) is secured to the upper elbow upper link (613a), towards a upper elbow proximal end (614a), such that rotation of the upper elbow (613) also facilitate rotation of the drive shaft (605).

9. The hand controller (207b) as claimed in claim 7, wherein a drive shaft (607) comprises a drive shaft upper end (607a) a drive shaft lower end (607b), and the drive shaft upper end (607a) is secured to the upper elbow lower link (613b), towards an upper elbow proximal end (614a), such that rotation of the upper elbow (613) will not facilitate rotation of the drive shaft (607).

10. The hand controller (207b) as claimed in claim 7, wherein a drive shaft (701) comprises a drive shaft upper end (701a) and a drive shaft lower end (701b), and the drive shaft lower end (701b) is secured to the lower elbow lower link (615b), towards the lower elbow proximal end (616a), such that rotation of the lower elbow (615) also facilitate rotation of the drive shaft (701).

11. The hand controller (207b) as claimed in claim 7, a pulley (707) is disposed towards the proximal end (614a) of the upper elbow (613) at the upper elbow lower link (613b) and a pulley (703) is disposed towards an upper elbow distal end (614b) of the upper elbow (613) upper elbow lower link (613b).

12. The hand controller (207b) as claimed in claim 11, a drive belt (705) is disposed from the pulley (707) to the pulley (703), and a drive shaft upper end (701a) is secured to the pulley (703), and the pulley (703) is rotationally connected to the drive belt (705) such that rotation of a drive shaft (701) along a axis of rotation ‘C’ will also facilitate rotation of the pulley (703) and (707) and thereby facilitate rotation of a drive shaft (607).

13. The hand controller (207b) of the console system (105) as claimed in claim 6, wherein the backend assembly (301) further comprising:
a first motorized joint assembly (403), wherein the first motorized joint assembly (403) is configured to facilitate movement of the hand controller (207b) in z-axis of a cartesian coordinates;
a second motorized joint assembly (405), wherein the second motorized joint assembly (405) is configured to facilitate movement of the hand controller (207b) in an x-axis of the cartesian coordinates; and
a third motorized joint assembly (407), wherein the third motorized joint assembly (407) is configured to facilitate movement of the hand controller (207b) in y-axis of the cartesian coordinates.

14. The hand controller (207b) of the console system (105) as claimed in claim 13, wherein the second motorized joint assembly (405) and the third motorized joint assembly (407) are configured to rotate along an axis of rotation ‘B’.

15. A first motorized joint assembly (403) of a hand controller (207b), comprising:
a plate (501);
a first pulley (503);
a first spring (505);
a first encoder assembly (602);
a first cable (506), wherein the first cable (506) circumscribes the first pulley (503) in a counterclockwise direction;
a first capstan (509), wherein the first capstan (509) is disposed tangential to a circumference of the first pulley (503);
a first motor (507), wherein the first motor (507) is secured to a posterior side of the plate (501); and
a counterbalance (527), wherein the counterbalance (527) is secured to a posterior side of the plate (501).

16. The first motorized joint assembly (403) as claimed in claim 15, wherein the plate (501) further comprises a plurality of holes (501a) and (501b) disposed at a horizontal end of the plate (501).

17. The first motorized joint assembly (403) as claimed in claim 16, wherein the hole (501a) is configured to accommodate a capstan (509).

18. The first motorized joint assembly (403) as claimed in claim 16, wherein the hole (501b) is configured to accommodate the first encoder assembly (602), a circular profile (503a) of the first pulley (503), in a same axis coaxially.

19. A second motorized joint assembly (405) of a hand controller (207b), comprising:
a second pulley (511);
a second spring (513);
a second encoder assembly (609);
a second cable (514), wherein the second cable (514) circumscribes the second pulley (511) in a counterclockwise direction;
a second capstan (517), wherein the second capstan (517) is disposed tangential to a circumference of the second pulley (511);
a coupler (601) having a first end (600a) and a second end (600b), wherein the first end (600a) includes a hole (601a), and the second end (600b) includes another hole (601b);
a second motor (515), wherein the second motor (515) is secured to the hole (601a) of the coupler (601); and
a drive shaft (605).

20. The second motorized joint assembly (405) as claimed in claim 19, wherein a circular profile (511a) is disposed to center of a diameter of the second pulley (511).

21. The second motorized joint assembly (405) as claimed in claim 19, wherein the second pulley (511) at its exterior surface around circumference includes a recess (512).

22. The second motorized joint assembly (405) as claimed in claim 19, wherein the second spring (513) is disposed within a recess (512).

23. The second motorized joint assembly (405) as claimed in claim 19, wherein the second cable (514) is secured to one end (513a) of the second spring (513) and the said second cable (514) circumscribes the second pulley (511) in counterclockwise direction and the second cable (514) wraps to the second capstan (517) several times and then secured to another end (513b) of the second pulley (511).

24. A third motorized joint assembly (407) of a hand controller, comprising:
a third pulley (519);
a third spring (521);
a third encoder assembly (611);
a third cable (522), wherein the third cable (522) circumscribes the third pulley (519) in a counterclockwise direction;
a third capstan (525), wherein the third capstan (525) is disposed tangential to a circumference of the third pulley (519);
a coupler (603) having a first end (604a) and a second end (604b), wherein the first end (604a) includes a hole (603a), and the second end (604b) includes another hole (603b);
a third motor (523), wherein the third motor (523) is secured to the hole (603a) of the coupler (603); and
a drive shaft (607).

25. The third motorized joint assembly (407) as claimed in claim 24, wherein a circular profile (519a) is disposed to center of a diameter of the third pulley (519).

26. The third motorized joint assembly (407) as claimed in claim 24, wherein the third pulley (521) at its exterior surface around circumference includes a recess (520).

27. The third motorized joint assembly (407) as claimed in claim 24, wherein the third spring (521) is disposed within a recess (520).

28. The third motorized joint assembly (407) as claimed in claim 24, wherein the third cable (522) is secured to one end (521a) of the third spring (521) and the said third cable (522) circumscribes the third pulley (519) in counterclockwise direction and the third cable (522) wraps to the third capstan (525) several times and then secured to another end (521b) of the third pulley (519).
